(19) United States
(12) Patent Application Publication
(10) Pub. No.: US 2010/0190133 A1
(43) Pub. Date: Jul. 29, 2010

(54) IRRIGATION AND ASPIRATION DEVICE

(76) Inventor: Daniel L. Martinez, Albuquerque, NM (US)

Correspondence Address: The Grafe Law Office, P.C., P.O. Box 2689, Corrales, NM, 87048, US

(21) Appl. No.: 12/753,057

(22) Filed: Apr. 1, 2010

Related U.S. Application Data
(63) Continuation-in-part of application No. 11/929,362, filed on Oct. 30, 2007.

(51) Int. Cl. A61C 5/02; A61C 17/08; A61M 1/00

(52) U.S. Cl. 433/81; 433/96; 604/319

(57) ABSTRACT

An irrigation and aspiration device comprises a probe and an aspiration sleeve that ensheathes the probe. The aspiration sleeve prevents the insertion of the probe beyond a desired distance, thereby preventing perforation of the apex of a root canal or wound, and also aspirates any excess irrigation solution and free debris. The device can be used for endodontic and periodontal procedures, penetrating or tract wound irrigation, open or superficial non-penetrating wound irrigation, and irrigation during specialty surgeries in endoscopic, ophthalmology, gynecology, orthopedic surgery, and other specialty surgeries.

### CROSS REFERENCE TO RELATED APPLICATIONS

This application claims priority as a continuation-in-part of U.S. application Ser. No. 11/929,362 filed Oct. 30, 2007, which is incorporated herein by reference.

### FIELD OF THE INVENTION

This invention is in the field of instruments for use in medicine and dentistry. More specifically, this invention is in the field of irrigator probes and syringes for irrigating root canals as part of a root canal procedure and for irrigating wounds in the field of trauma and surgery.

### BACKGROUND OF THE INVENTION

To preserve a tooth that has a diseased pulp cavity, it is necessary to prevent bacterial proliferation within the pulp canal of the tooth by removing the diseased or necrotic pulp material from the pulp canal. After the pulp material has been removed or extirpated from a tooth, the pulp cavity is typically filled or obturated with a material such as gutta percha to occlude the pulp cavity and a viscous sealer to aid in sealing the pulp cavity. This procedure is referred to as root canal therapy. Root canal cleaning is generally achieved by hand or mechanical instrumentation with files that are configured to bore and cut.

It is also common during the root canal procedure to irrigate a pulp cavity and the various root canals involved using an endodontic irrigator tip. Irrigation assists in removing debris and necrotic material cut by the endodontic files and reamers. Disinfecting solutions can also be employed in irrigation, thereby disinfecting the pulp cavity and root canals during the operative procedure.

In addition to being thin and tight, root canals are often twisted and cumbersome to negotiate. In order to place irrigator tips at a convenient angle within a tooth and negotiate the convoluted passages of root canals, irrigator tips are typically bent. U.S. Pat. No. 4,993,941 to Maita et al., for example, discloses a dental irrigator needle having a selectively angled cannula that was designed to provide more convenient fluid delivery. U.S. Pat. No. 5,127,831 to Bab also describes a bendable flexible irrigation probe.

Another problem associated with root canal therapy is apical perforation. Before a file or irrigation tip is inserted into a root canal, the length of the root canal is determined to identify a suitable working length for the file or irrigation tip. Generally, the working length corresponds to the distance from a fixed reference position on the crown of a tooth to a location above the apical constriction of the root canal. Radiography is the most common method for measuring the length of the root canal. The preoperative x-ray image of the diseased tooth is taken from the front or back of the tooth. The length of the root canal and the desired working length of the cannula to be placed therein are then determined.

Perforation of the apex of a root canal can result from the use of files or endodontic irrigation tips that are too long. Such apical perforations typically result from an error in estimating the length of a root canal or the working length of the cannula. Similarly, the apex can be perforated by extrusion of infected material through the apex due to the force exerted by the file or tip on the material as the file or tip is pushed downward to reach the apex. In addition to exposing the tissue surrounding the tooth to the infected material, apical perforations also substantially complicate subsequent healing of the root canal.

The possibility of perforating the apex is particularly frustrating because it is often desirable to deliver fluid that reaches the apex in order to disinfect the apex and dissolve necrotic tissue therein. However, certain fluids are too viscous or the surface tension prevents certain fluids from reaching the apex if delivered too far above the apex. Sodium hypochlorite, for example, is a widely used, strong disinfectant that, because of surface tension issues can stick in the pulp chamber rather than reaching the apex if not delivered with precision from the appropriate location above the apex. Moreover, to truly irrigate necrotic debris it is often advisable to have the irrigant be delivered distal to the debris. Attempts to deliver the solution from the appropriate location, however, may result in the perforation of the apex.

Practitioners have made some attempts to place irrigator tips within teeth without perforating the apex. The working length of endodontic irrigator tips is sometimes limited by (i) bending the tips to prevent them from extending too far into a root canal, or (ii) marking the tips with a pen at a location on the tips which should not extend past the rim of the crown. The problem with each of these approaches, however, is that they are unreliable, and can contaminate the tip as a result of the handling of the tip prior to use. In addition, bending a cannula can crimp or kink the cannula.

The possibility of perforating the apex of the root canal with an endodontic file is sometimes prevented by employing a removable stop that is placed about the distal insertion end of the file and pushed a desired distance toward the proximal gripping end of the file. Such adjustable stops, however, are prone to slip and slide along the longitudinal axis of the file, thereby allowing perforation of the apex. In addition, placing a stop on the file requires handling of the file prior to use, possibly contaminating the file. One solution to this was the use of a movable collar or neck on the irrigation probe as described in U.S. Pat. No. 6,079,979 to Riitano. This probe is similar to the probe described in U.S. Pat. No. 6,422,865 to Fisher, again without a suction or aspirating portion. However, this movable collar does not address the problem of the caustic irrigant solution as described below.

One of the greatest complications of endodontic root canal irrigation is leakage of the irrigant fluid, which is often sodium hypochlorite or other caustic antiseptic solution, onto the mucosa of the mouth (gums, gingivae, etc). For example, injection of the irrigant into the periapical tissues can cause necrosis of these tissues, i.e., bone and periodontal ligament, causing a severe and painful chemical burn. Often an operator must put the aspirating tip of a surgical vacuum handle directly on the surface of the crown next to the irrigator needle in order to aspirate the waste irrigant solution before it spills onto the oral mucosa and causes a severe chemical burn. For practitioners that do not use sterilizing irrigant, but rather use water or normal saline, the greatest danger is splash-back onto the operator, increasing their danger of acquiring hepatitis, HIV, and other infectious diseases. Thus, simultaneous aspiration is important in this instance also. This is a very cramped environment with both the irrigating syringe and probe and the surgical aspiration vacuum handle in the patient's mouth. U.S. Pat. Appl. No. 2006/0259014 to Yarger describes a typical aspirator sleeve and handle for this purpose. U.S. Pat. No. 4,272,288 to Yoshii et al. describes an irrigation pen that permits continuous irrigation of a root canal, but no simultaneous aspiration.

U.S. Pat. No. 3,807,048 to Malmin describes an endodontic gun that can inject, irrigate, and evacuate for this purpose, but it is not a simple syringe technology, involves the use of valves, and can only sequentially irrigate and aspirate, rather than simultaneously irrigate and aspirate. U.S. Pat. Nos. 5,203,697, 5,490,779, and 5,540,587 to Malmin also describe an endodontic device that can aspirate and inject, but again sequentially, rather than simultaneously. U.S. Pat. No. 4,993,947 to Grosrey demonstrates an irrigation needle surrounded by a vacuum sheath, but the device does not use a syringe for the irrigant solution nor does the vacuum sheath move along the shaft of the needle.

U.S. Pat. No. 3,871,099 to Kahn discloses a device with 1) a syringe, 2) an endodontal irrigation needle, and 3) plastic tubing of two different diameters that function as a vacuum sheath. To construct this device, a needle is inserted at angle through the larger tubing, and after penetrating the larger tubing the needle is bent to become collinear with the long axis of the internal lumen of the both the small tubing and large tubing while the syringe held at an angle to the collinear lumen (FIGS. 1 and 2 of U.S. Pat. No. 3,871,009). Note that in Kahn the needle is collinear along much, but not all, of the portion of the needle within the tube. There is a sharp bend inside the tube near where the needle enters the tube; this sharp bend makes it difficult to effectively change the projection of the needle out of the tubing by moving the needle into or out of the tubing. The angular bending of the needle fixes the length of the vacuum sheath relative to any point along the needle shaft and also fixes the position of the distal end of the vacuum sheath relative to the distal end of the needle. Thus, U.S. Pat. No. 3,871,009 controls the relationship of the distal end vacuum sheath to the distal needle tip, the depth of the needle into the root canal, and the angle of the syringe to the larger tubing and distal needle shaft by bending the needle internal to the tubing at the large tubing-small tubing junction. In terms of durability, safety and needle control, when the needle is bent close to the hub of the needle as in U.S. Pat. No. 3,871,009 greater stresses deformity occur at the needle hub-needle junction, and fracture of the shaft of needle just above the hub or mechanical kinking of the needle causing obstruction are more likely. These mechanical stresses are multiplied as the bent portion of the needle moves in and out of the stiffer larger tubing if the vacuum sheath is adjusted in depth in relation to the end of the needle, or the needle must be straightened and rebent at another portion of the needle shaft. This requires forceful movements along the bent angle of the needle with sudden giving as the bent portion of the needle moves in and out of the tightly adherent and constrained penetration hole through larger tubing, all of which causes dangerous loss of control of the needle tip in the mouth and endodontal cavity, increasing the risk of perforation of the apex of the tooth as well as fracture of the needle. Thus, there is a need for a device where the aspirating vacuum sheath can be easily and smoothly adjusted without a need for rebending the needle and without catching or stopping.

Manufacturability, low-cost assembly, and reduction of hand-assembly are important in the mass production of cost-effective devices. In U.S. Pat. No. 3,871,009 the device has to be extensively assembled by hand including 1) placing the flexible smaller distal tubing into the larger tubing, 2) piercing the larger or smaller tubing with the needle, 3) threading the needle down into the smaller tubing, and 4) bending the needle. This requires expensive hand assembly and is dangerous to the assembly worker in that the fingers can be pierced by the needle during assembly or, if the sheath alone is supplied, the danger of piercing the fingers of the operator is considerable. The devices of U.S. Pat. No. 3,871,009 were difficult to mass-manufacture due to the extensive hand assembly required and the constant danger of needlestick when inserting the irrigation probe through the wall of the vacuum tubing while holding the vacuum tubing in the fingers. Thus, there is a need for an irrigation syringe-vacuum sheath combination that can be both 1) machine-assembled at high volumes and low cost with minimal hand assembly, and 2) assembled with a reduced risk of finger penetration or needlestick to the assembly worker and to the operator while providing similar or enhanced function.

There are a number of irrigation handles that substitute for syringe irrigation. U.S. Pat. No. 6,464,498B2 and US 2002/0142260A1 to Pond describes reusable aspirating and irrigating handle consisting of a hand piece, non-conventional I-Shaped needle, and a L-shaped fluid connector that supports the needle. In the Pond device a fluid source is provided by the L-shaped fluid connector, and delivered to the tooth by gravity and surface tension, while vacuum is provided by the needle. Moreover, the L-shaped connector is not movable along the needle shaft, but is fixed to the syringe body, thus, the distance of the needle tip to the connector is fixed, and the only method to adjust relationship of the distal needle tip to the distal end of the L-shaped fluid fitting is to trim either the needle or trim the L-shaped fitting. Similarly, U.S. Pat. No. 7,270,544 to Schemmer demonstrates an L-shaped irrigation handle with an aspirating and injection function, but no sheath and the L-shaped portion is fixed and does not move along the shaft of the needle. U.S. Pat. No. 4,215,476 to Armstrong demonstrates a simultaneous aspiration-injection irrigation handle with an L-shaped vacuum sheath, but this sheath is fixed and is not moveable along the needle shaft, and does not use a syringe as the fluid source. U.S. Pat. Nos. 5,913,859 and 6,846,314 to Shapira also demonstrate a handle device, with an L-shaped vacuum sheath for bone marrow extraction; however, this sheath is also fixed to a handle, and is not moveable along the axis of the bone marrow drill, and this is not an irrigation device. US 2009-004621A1 to Quan demonstrates a irrigation handle with an irrigation needle surrounded by a vacuum sheath; however, the sheath is fixed in relation to the shaft of the needle and the device employs a non-standard irrigation needle. A problem with handle devices in general are that they are expensive to manufacture, do not permit different needle and/or sheath depths, and do not use standard irrigation probes or standard syringes, and thus are relatively expensive and inflexible.

U.S. Pat. No. 6,641,394 to Garman demonstrates a simultaneous aspiration-irrigation syringe device, but in this device the vacuum source is a syringe, not a conventional dental vacuum, and the irrigation source is not surrounded by a vacuum sheath, nor are standard syringe and standard irrigation needle are used in the device.

US 20070244425 to Pond shows an irrigation device with a vacuum sheath, irrigation needle, and syringe. However, although the device can use a standard syringe, the vacuum sheath is firmly fixed to the syringe, and is not movable along the needle shaft, the needle does not pierce tubing, the vacuum seal is not between the needle shaft and sheath, but between the sheath and syringe, the needle is not a standard irrigation needle, and either the sheath or the needle must be trimmed with scissors to adjust to depth within the root canal.

Thus, there is a need for an irrigation-aspiration syringe that can deliver irrigant solution to the apex of the tooth and simultaneously aspirate necrotic debris and spent irrigant solution before it burns the mouth or splashes back on the operator while at the same time permitting moment to moment adjustment of probe depth in the tooth and sheath position on the probe while using standard irrigation needles and conventional syringes if desired. This device preferably is amenable to low-cost mass production, requires minimal hand assembly, and should have a low risk of accidental needlestick while inserting the irrigation probe into the sheath.

Analogous to an infected root canal, deep penetrating wounds (human and animal bites, stab wounds, penetrating wounds from a wooden branch, etc), must be irrigated to remove foreign material, necrotic debris, infection, and pus. Typically a rigid or flexible cannula is placed into the wound and the wound is irrigated with normal saline. The position of the probe or cannula is important so that further penetration into normal tissue does not occur, thus, a mechanism to keep the cannula at the correct level is important. Irrigation of a penetrating wound is a very messy procedure, and the irrigant solution often splashes back at the operator and assistant, increasing their danger of acquiring hepatitis, HIV, and other infectious diseases. Thus, simultaneous aspiration at the wound surface would be advantageous for penetrating wounds also.

Open or superficial wounds, similarly to penetrating wounds, must also be irrigated to remove foreign material, necrotic debris, infection, and pus. Although the position of the irrigation probe or cannula is not as critical as in endodontic root canals or penetrating wounds, the procedure for superficial wounds is also very messy with extreme splash back, again endangering the operator and assistant. U.S. Pat. No. 5,941,859 to Lerman discloses a splash shield, aspiration port, and irrigation probe that permit both aspiration and irrigation of superficial or flat wounds; however, the aspiration and irrigant ports are not collinear or one residing within the other. This type of technology would not function for root canals or deep penetrating wounds. US 2006/0292522 to Lees discloses a splatter shield for a spinning dental brush used to polish tooth surfaces. The Lees shield passively blocks splatter towards the operator but does not block splatter towards the patient, is not part of a vacuum sheath, does not aspirate spatter, is not used for irrigation, and does not move axially and reversibly along the instrument shaft, thus, it is not adjustable as to depth.

US06045516 to Phelan demonstrates a stylet used to place an aspiration catheter. The stylet is not intended to place a soft irrigator catheter with a suction sheath into deep wounds and then be removed so that fluid can be irrigated down the catheter, rather the stylet of this device is intended to be used to unclog aspirator device when it becomes obstructed during use, like cleaning the barrel of gun.

One type of suction catheter is disclosed in U.S. Pat. No. 3,375,828 to Sheridan in which a rolled up sleeve can be unrolled over the airway control aperture to apply suction in the nose, mouth, pharynx, trachea, bronchi, or other cavity in the body of a patient. In U.S. Pat. No. 3,982,540 to Ross, a multilumen tube is disclosed in which there are a plurality of spaced suction apertures in the outside wall of the negative pressure tube and a plurality of spaced positive pressure apertures, each located behind a suction aperture, to dislodge particles blocking the suction apertures. In U.S. Pat. No. 4,014,333 to McIntyre, a combined irrigation and aspiration instrument is disclosed for use in ophthalmic surgery wherein an inner and an outer tube of straight rigid material are provided in fixed relationship, the suction tip of the inner tube projecting beyond the irrigation tip of the outer tube, so that the debris-receiving, suction inlet opening is in front of, and beyond the annular pressurized irrigation outlet opening. The above mentioned Ross and McIntyre patents, which disclose combined suction and irrigation, both teach the placement of the pressurized liquid outlets in rear of the suction inlets and both teach a fixed relationship of the irrigation tube and the suction tube, so that the irrigation tube cannot be easily and quickly removed.

In addition to the above patents, there is a line-of-road vacuum cleaning apparatus, typified in the patents listed below in which there are debris-receiving inlets and/or suction inlets combined with air pressure outlets for dislodging debris. However, these patents also teach placing the air outlet in rear of, or flush with, the suction, or debris-receiving, inlet, including U.S. Pat. No. 2,990,019 to Finn, U.S. Pat. No. 3,221,358 to Dickson, and U.S. Pat. No. 3,447,188 to Maasbery. U.S. Pat. No. 5,447,494 to Dorsey, U.S. Pat. No. 5,573,504 to Dorsey, and U.S. Pat. No. 4,468,216 to Muto attempted to address this problem, by providing a catheter that simultaneously irrigates and aspirates by means of an irrigation cannulae residing within a suction cannula with or without valves; however, these provide only point irrigation-suction which is not optimal for root canal or deep penetrating wounds, the suction sleeve is not adjustable, does not provide continuous suction, does not prevent perforation, and does not provide splash back protection at the surface of the tooth or wound. U.S. Pat. Nos. 6,394,996 and 6,878,142 to Lawrence et al. disclose a catheter for irrigating tract wounds with or without a splash shield. However, the suction source is not adjustable and is parallel to the irrigation cannula rather than being a sleeve, and is a complex gun set up, rather than a simple syringe with vacuum. U.S. Pat. No. Des. 365146 to Olson discloses a wound debridement tip with an aspirator sleeve, but the sleeve is not adjustable, and the device cannot be used on a conventional syringe. Thus, although an improvement, these devices do not provide the qualities needed for root canal or wound irrigation.

The prior art does not reveal, and there is therefore a need for an irrigation probe and syringe that can be economically manufactured with minimal hand-assembly and can be reliably positioned to prevent distal perforation of the root canal or wound and can irrigate with saline, water, or caustic solutions while simultaneously aspirating the spent irrigant solution and necrotic debris, preventing chemical burns to surrounding tissues, and minimizing hazardous splash back at the operator while permitting precise intraoperative adjustments of needle and/or sheath/shield depth.

### SUMMARY OF THE INVENTION

An endodontic or periodontic irrigation and aspiration device comprises a conventional or non-conventional syringe, an irrigation probe of various designs and manufacturers, and a movable aspirating sleeve in which the irrigation probe resides. The aspirating sleeve is adjustable on the length of the irrigation probe so that different depths of root canals or periodontal recesses can be irrigated with the sleeve aspirating on or within the crown of the tooth or the mucosal surface of the periodontal tissues, respectively. The aspirating sleeve can be a one piece device comprising a body and a fitting that permits a vacuum source in the form of vacuum tubing to be attached, a portion that permits an needle to be inserted into the sleeve along a substantially straight line, and an aspirating portion comprising an elongated portion in which the irrigation probe or needle resides. Alternatively, the aspirating sleeve can comprise several components which are then assembled to the same effect. The vacuum tubing can have a proximal fitting, similar to a surgical vacuum handle, to permit access to conventional dental suction vacuum equipment so that the device can be immediately applicable to all dental, periodontal, and endodontic offices and practices. Because the device can be mass produced without extensive or dangerous hand assembly, the cost of the device is low and the device is completely disposable. The cost of the device is also low because conventional irrigation probes and syringes can be used if desired. The irrigation and aspiration device permits effective root canal irrigation and can prevent caustic irrigation solution from damaging the mucosa of the mouth.

A penetration or tract wound irrigation and aspiration device comprises a conventional or non-conventional syringe, wound irrigation probe of various designs and manufacturers, and an aspirating sleeve in which the irrigating probe resides. The aspirating sleeve can be adjustable on the length of the irrigation probe so that different depths of penetrating or tract wounds can be irrigated with the sleeve aspirating on the skin surface to prevent splash-back on the operator. The aspirating sleeve can be plastic vacuum tubing that is penetrated by the irrigation probe and movable on the shaft of the probe or can be a dedicated movable sleeve with a proximal fitting to permit attachment of vacuum tubing. A splash shield can be integrated into the vacuum aspiration sleeve. The vacuum tubing can have a proximal fitting, similar to a surgical vacuum handle or medical vacuum tubing, to permit access to conventional medical suction vacuum equipment so that the device can be immediately applicable to all medical and hospital offices and practices.

An open or superficial wound irrigation and aspiration device comprises a conventional or non-conventional syringe, wound irrigation probe of various designs and manufacturers, and an aspirating sleeve in which the irrigating probe resides. The aspirating sleeve can be fixed on the length of the irrigation probe and comprises a rigid or flexible splash shield with the sleeve aspirating on the wound surface to prevent splash-back. A flexible vacuum splash shield permits suction to be applied across the wound surface permitting better debridement and greater splash protection. The aspirating sleeve can be plastic vacuum tubing that can be penetrated by the irrigation probe and movable on the shaft of the probe or can be a dedicated movable sleeve with a fitting to permit attachment of vacuum tubing. A splash shield can be integrated into the vacuum aspiration sleeve. The vacuum tubing can have a proximal fitting, similar to a surgical vacuum handle or medical vacuum tubing, to permit access to conventional medical suction vacuum equipment so that the device can be immediately applicable to all medical and hospital offices and practices.

Kits featuring different irrigation probes, such as probes having cannulae with a variety of different working lengths and diameters and designs with an adjustable aspirating sleeves and various sizes and types of syringes and irrigation sources can be provided, thereby enabling the practitioner to treat root canals and wounds of varying sizes and depths and irrigant volume requirements.

### BRIEF DESCRIPTION OF THE DRAWINGS

The accompanying drawings, which are incorporated in and form part of the specification, illustrate the present invention and, together with the description, describe the invention. In the drawings, like elements are referred to by like numbers.

FIG. 1 is a schematic illustration of an irrigation probe with an aspiration sleeve comprising a single piece attached to vacuum tubing appropriate for endodontic and periodontal irrigation.

FIG. 2 is a schematic illustration of an irrigation probe with an aspiration sleeve comprising a dedicated vacuum housing with fittings for vacuum tubing.

FIG. 3 is a schematic illustration of an irrigation probe with an aspiration sleeve incorporating a movable splash shield most appropriate for penetrating or tract wound irrigation.

FIGS. 4A and 4B are schematic illustrations of an irrigation probe with a removable stylet and aspiration sleeve with or without movable splash shield most appropriate for inserting a flexible irrigation probe into a penetrating or tract wound.

FIG. 5 is a schematic illustration of an irrigation probe or cannula with an aspiration sleeve incorporating immovable, but flexible or rigid, splash shield most appropriate for open or superficial wounds.

FIGS. 6A-6C are schematic illustrations of an irrigation probe with an aspiration sleeve and different irrigant sources.

FIG. 7 is a schematic illustration of an irrigation probe and aspiration sleeve with a double barrel reciprocating syringe that simultaneously provides injection and aspiration.

FIGS. 8A-8C are schematic illustrations of an irrigation probe showing a movable aspiration sleeve in different positions relative to the tip of the irrigation probe.

FIG. 9 is a schematic illustration of a typical kit having an irrigation probe and aspiration sleeve comprising various diameters of vacuum tubing, syringe, and vacuum fitting most appropriate for endodontic and periodontal irrigation.

FIG. 10 is a schematic illustration of an irrigation probe and aspiration sleeve comprising various diameters of vacuum tubing, and syringe showing irrigation and aspiration of caustic irrigant solution typically used in endodontic irrigation.

FIG. 11 is a schematic illustration showing how an irrigation syringe can be operated with one hand while the aspirating sleeve is controlled with the other hand.

FIG. 12 is a schematic illustration how the aspirating sleeve can be comprised of one piece of tubing in a controlled one-piece frame.

### DETAILED DESCRIPTION OF THE INVENTION

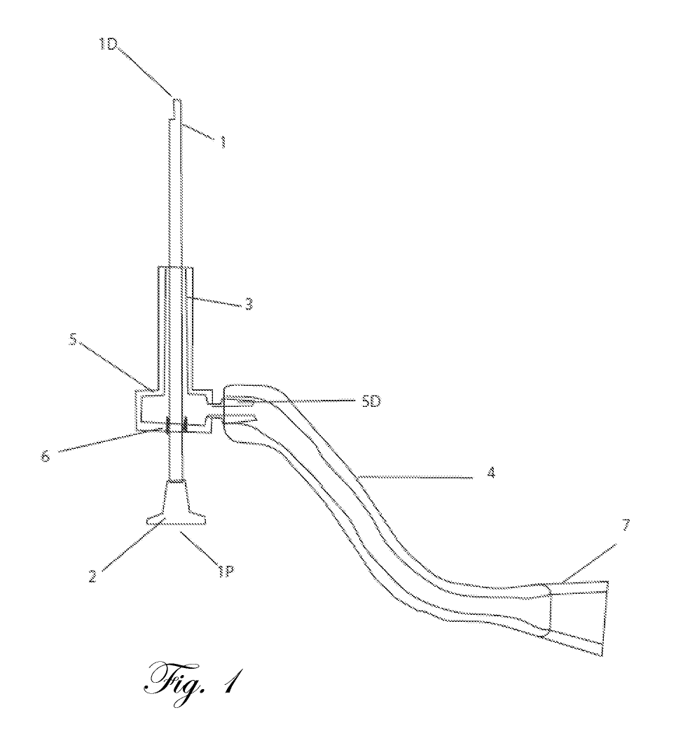

With reference now to FIGS. 1 to 12, example irrigation and aspiration devices of the present invention are described. The irrigation and aspiration device comprises an irrigation probe, or cannula, and an aspiration sleeve that ensheathes the probe. The device has a distal end and a proximal end. The aspiration sleeve can prevent the insertion of the distal end of the probe beyond a desired distance, thereby preventing perforation of the apex of a root canal or wound, and also aspirates any excess irrigation solution and free debris. The distal stop end of the aspiration sleeve can have a substantially greater diameter than the diameter of the distal end of the probe. Thus, the stop end of the sleeve can rest on the occlusal surface of the crown of a tooth or wound while the distal end of the probe extends the desired distance within the root canal or wound. The irrigation and aspiration device can be used for convenient delivery of a variety of different liquids to a root canal, periodontal recess or wound, such as debriding agents, lubricants, anti-bacterial agents, chelating agents, water, hypochlorite, ablating agents, alcohol, contrast, or other injectables. The aspirating sleeve is designed for mass production with minimal hand assembly, especially important in avoiding puncture wounds that occur with other vacuum sleeve designs.

FIG. 1 is a schematic illustration of an irrigation probe with an aspiration sleeve, or sheath, comprising a single-piece body and a diameter of vacuum tubing most appropriate for endodontic and peridontal irrigation. The device has a distal end and a proximal end. Irrigation probe 1 can be a rigid, bendable, or flexible irrigation needle, catheter, cannula, or similar probe and an appropriate fitting 2 at the proximal end 1P for a syringe or other fluid source to deliver irrigant from a tip at the distal end 1D of the probe. Irrigation probe 1 is ensheathed within a T-shaped aspiration sleeve 3 which can be a flexible or rigid hollow sheath for vacuum aspiration. The present invention includes embodiments wherein the aspiration sleeve can be T-shaped, L-shaped, Y-Shaped, or other configurations that provide similar connectivity and funcationality. The aspiration sleeve 3 can be collinear with the shaft of the probe 1. The aspiration sleeve 3 can be continuous with or connected to vacuum tubing 4 by an air-tight bond 5 (here shown as a single-injected molded piece), and can be movable on the shaft of the probe 1 with a contact point at a near or substantially air-tight junction 6 so that vacuum can be diverted to tip 1D. The air-tight junction 6 can comprise a through opening in the wall of the sheath or can be a membrane or surface that can be penetrated by a needle or cannula. The substantially air-tight junction 6 thereby enables the probe 1 to slide and change positions relative to the aspiration sleeve 3. The vacuum tubing 4 can be connected to the aspiration sleeve by a fitting 5D and to a vacuum source by means of a proximal vacuum fitting 7. Both of these fittings can be of various and complementary designs so that they reversibly bond to the tubing, or can be cemented, welded, or injection molded so that there is a vacuum-tight seal. The vacuum tubing 4 can be constructed of Tygon, but can be of any flexible polymer or plastic. Similarly, the aspiration sleeve 3 can be constructed of Teflon tubing, but can be constructed of Tygon, or any biocompatible polymer or plastic or other flexible or rigid material. This device can provide similar function to that shown in U.S. Pat. No. 3,871,009 to Kahn, but with this embodiment the movement of the sheath on the probe is smoother because the needle can be bent distally instead of proximally, and the risk of accidental needlestick from the probe during assembly is markedly reduced by the use of a frame that permits the vacuum sleeve to be held firmly while the fingers are protected by the frame. Unlike U.S. Pat. No. 3,871,009 to Kahn, this device can be mass-produced inexpensively with minimal hand assembly while reducing assembly injuries.

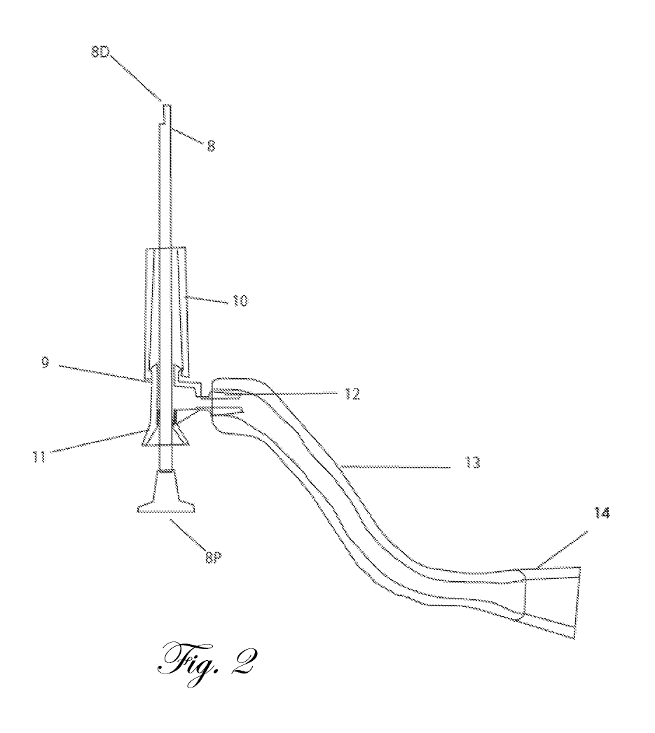

FIG. 2 is a schematic of an irrigation probe with an aspiration sleeve comprising a dedicated vacuum housing with fittings for vacuum tubing. Irrigation probe 8 can be a rigid, bendable, or flexible irrigation needle, catheter, cannula, or similar probe with an appropriate fitting at the proximal end 8P for a syringe or other fluid source to deliver irrigant from a tip at the distal end 8D of the probe. Irrigation probe 8 resides in and can be collinear with a hollow aspiration sleeve 10 for vacuum aspiration. The aspiration sleeve 10 can be flexible and can be part of or connected to the vacuum housing 11 with an air-tight bond 9 which connects the sleeve to the housing. The vacuum housing with a funnel shaped port 11 comprises a fitting 12 attached to vacuum tubing 13 and can be movable on the shaft of the probe 8 with a contact point at a near or substantially air-tight junction. The vacuum tubing 13 can be connected to a vacuum source by means of a fitting 14. This device can provide similar function to that shown in U.S. Pat. No. 3,871,009 to Kahn, but with this embodiment the movement of the sheath on the probe is smoother because the needle can be bent distally, and the risk of accidental needlestick from the probe during assembly is markedly reduced by the funnel shaped probe port 11 that provides controlled introduction of the needle into the sheath and the use of a housing that permits the sheath to be held firmly while the fingers are protected by the housing. Unlike U.S. Pat. No. 3,871,009 to Kahn, this device can also be mass-produced inexpensively with minimal hand assembly.

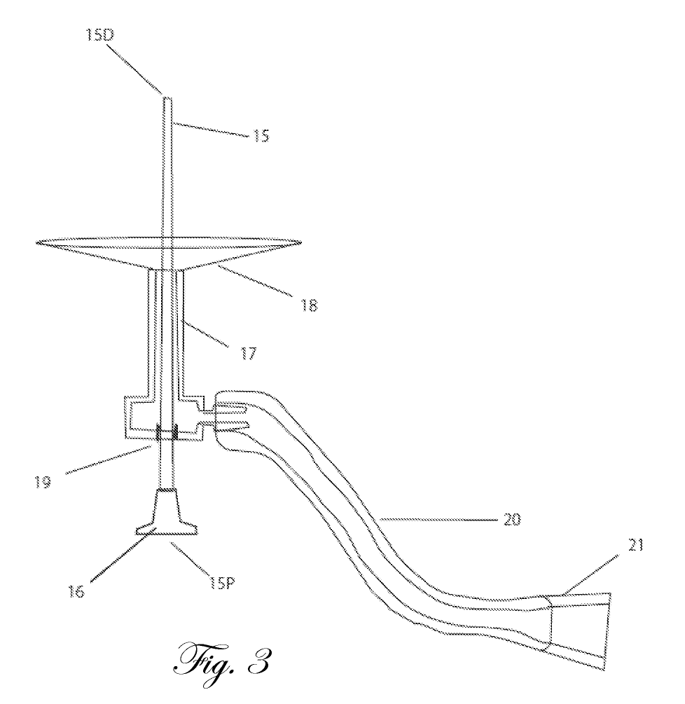

FIG. 3 is a schematic illustration of an irrigation probe with an aspiration sleeve incorporating a movable splash shield appropriate for penetrating or tract wound irrigation. Irrigation probe 15 can be a rigid, bendable, or flexible irrigation needle, catheter, cannula, or similar probe with an appropriate fitting 16 at the proximal end 15P for a syringe or other fluid source to deliver irrigant from a tip at the distal end 15D of the probe. Irrigation probe 15 resides in and can be collinear with an aspiration sleeve 17 that has a flexible or rigid vacuum splash shield 18. As in FIGS. 2 and 3, there is a near or substantially airtight junction 19 between the probe 15 and housing or vacuum tubing 20, and a fitting 21 to attach to a vacuum source. The splash shield 18 can comprise a conical, hemispherical, or other geometric structure of rigid or flexible plastic material, preferably nearly or completely transparent. Splash shield 18 and sleeve 17 can be movable on the shaft of the irrigation probe 15, so that the splash shield 18 can remain on the surface of the skin while the irrigation probe 15 can be inserted to different depths in the penetrating or tract wound.

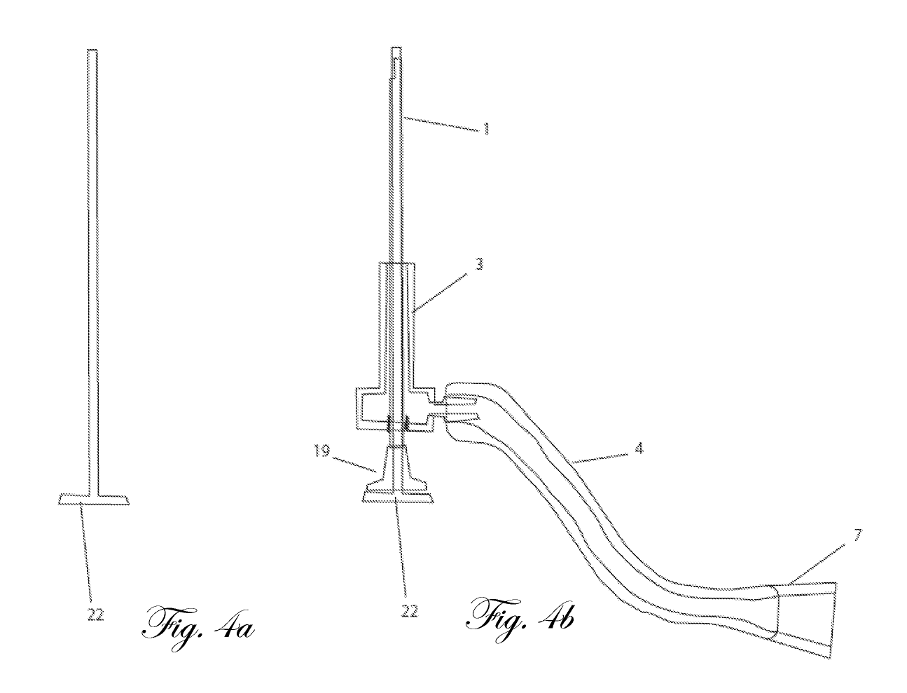

FIGS. 4A and 4B are schematic illustrations of an exemplary irrigation probe 1 and aspiration sleeve 3 of the type shown in FIG. 1, with or without a movable splash shield, and a removable stylet 22, as shown in FIG. 4A. This device is appropriate for inserting a flexible irrigation probe into a penetrating or tract wound. As shown in FIG. 4B, the stylet 22 can be placed in the flexible irrigation probe 1, directed into the tract or penetrating wound to the correct depth, and then can be removed. The irrigation probe 1 can then be attached to an irrigant fluid source with a fitting 2.

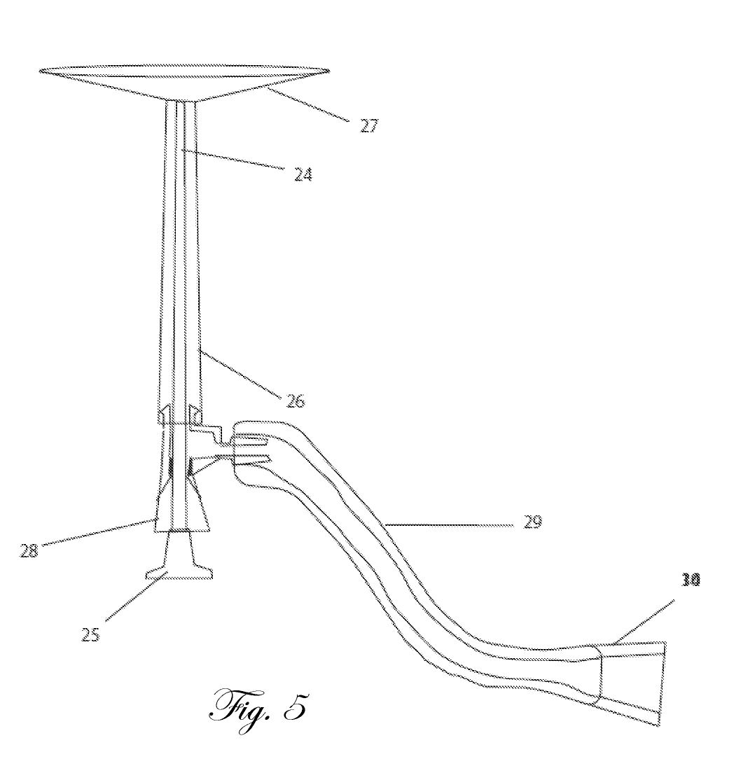

FIG. 5 is a schematic illustration of an irrigation probe with an aspiration sleeve incorporating an immovable, but flexible or rigid, splash shield most appropriate for open or superficial wounds. Irrigation probe 24 can be a rigid, bendable, or flexible irrigation needle, catheter, cannula, nozzle, or similar probe with an appropriate fitting 25 for a syringe or other fluid source. Irrigation probe 24 resides in a hollow aspiration sleeve 26 that has a flexible or rigid vacuum splash shield 27. There can be an air-tight junction 28 and support between a housing or vacuum tubing 29, and a fitting 30 to attach to a vacuum source. Vacuum splash shield 27 and sleeve 26 can be immovable on the shaft of the irrigation probe 24, so that the splash shield 27 can remain on the surface of the skin.

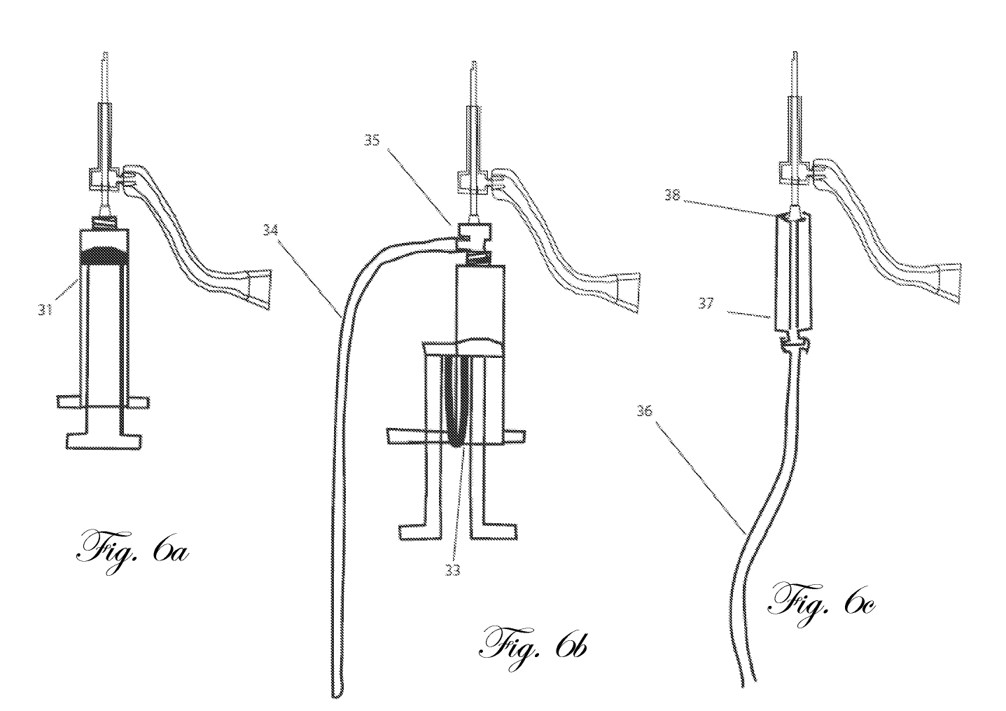

FIGS. 6A to 6C are schematic illustrations of an exemplary irrigation probe and aspiration sleeve as shown in FIG. 1 with different irrigant sources demonstrating the flexibility of this type of aspiration device. FIG. 6A shows the irrigation probe with a conventional syringe 31. FIG. 6B shows the irrigation probe with a refilling syringe 33 and fluid tubing and fluid source 34 with double one-way valves 35 to permit refilling of the syringe. Syringes as described in U.S. Pat. No. 6,245,046, U.S. Pat. No. 6,962,576, U.S. Pat. No. 7,118,554, and U.S. Pat. Appl. No. 2006-18410-A1 to Sibbitt et al. are examples of such refilling syringes. FIG. 6C shows a continuous or adjustable fluid source 36 in a handle or pen 37 with on-off valve 38, similar to the handle described in U.S. Pat. No. 4,272,288 to Yoshii et al.

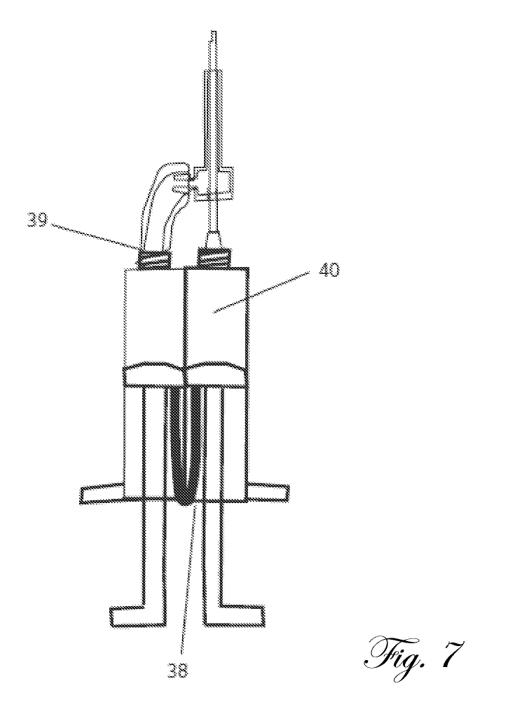

FIG. 7 is a schematic illustration of an irrigation probe and aspiration sleeve with a double barrel reciprocating syringe 38 that simultaneously provides for irrigant injection and aspiration. In this embodiment, the vacuum tubing or vacuum housing is provided vacuum by attaching to the accessory barrel needle fitting 39 of the double barrel reciprocating syringe 38, while the irrigation probe is attached to the needle fitting to the injection barrel 40 of the reciprocating syringe. Syringes as described in U.S. Pat. No. 6,245,046, U.S. Pat. No. 6,962,576, U.S. Pat. No. 7,118,554, and U.S. Pat. Appl. No. 2006-18410-A1 to Sibbitt et al. are examples of such reciprocating syringes. Each of the foregoing is incorporated herein by reference.

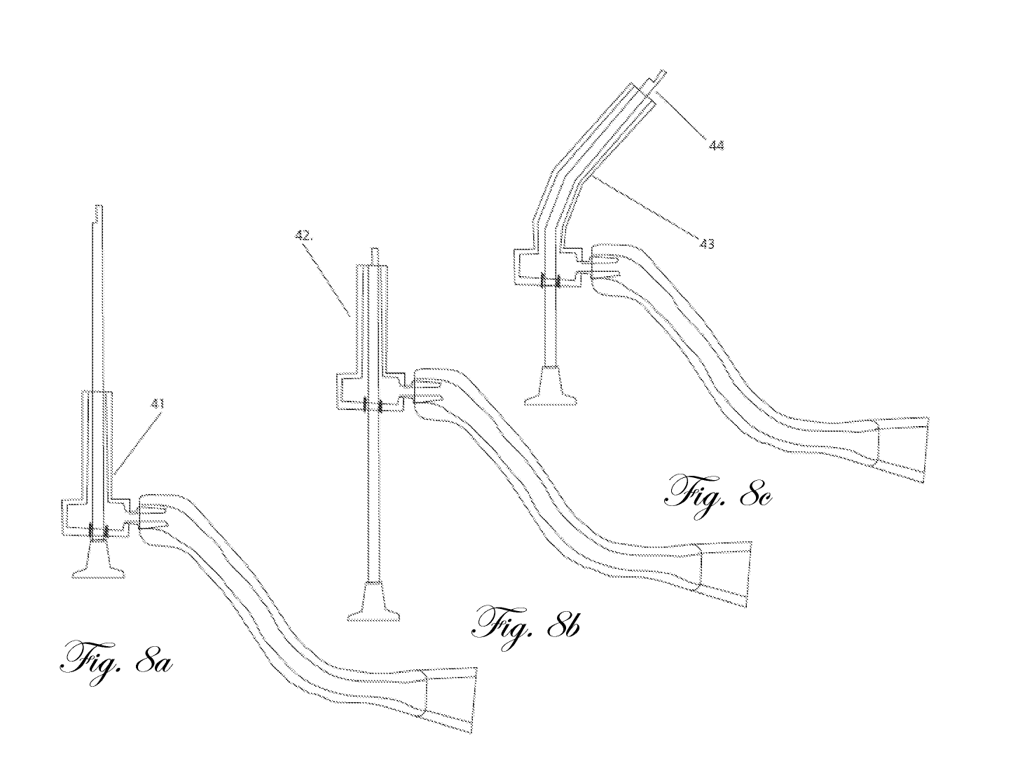

FIGS. 8A to 8C are schematic illustrations of an irrigation probe showing a movable aspiration sleeve in different positions relative to the tip of the irrigation probe by movable and reversible sliding of the sleeve along the shaft or the straight or bent needle or probe. FIG. 8A shows the aspiration sleeve in a retracted position 41 as would be used for a deep root canal or wound irrigation. FIG. 8B shows the aspiration sleeve in an extended position 42 for a shallow irrigation. FIG. 8C shows the aspiration sleeve in a fixed position 43 around an intentionally bent needle or probe 44.

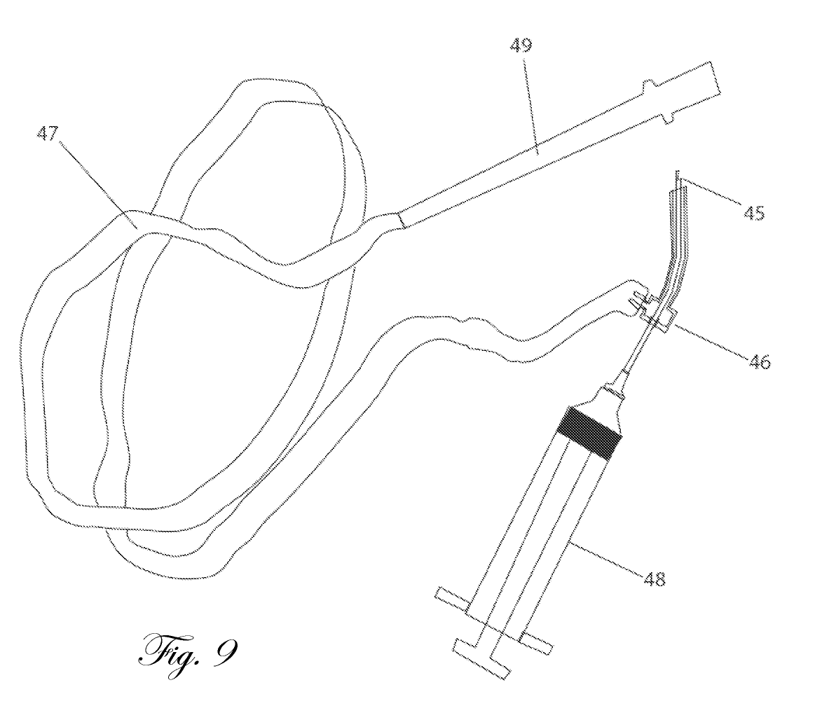

FIG. 9 is a schematic illustration of a typical kit with irrigation probe 45 and aspiration sleeve 46 comprising various diameters of vacuum tubing 47, irrigation syringe 48, and vacuum fitting 49 most appropriate for endodontic and peridontal irrigation.

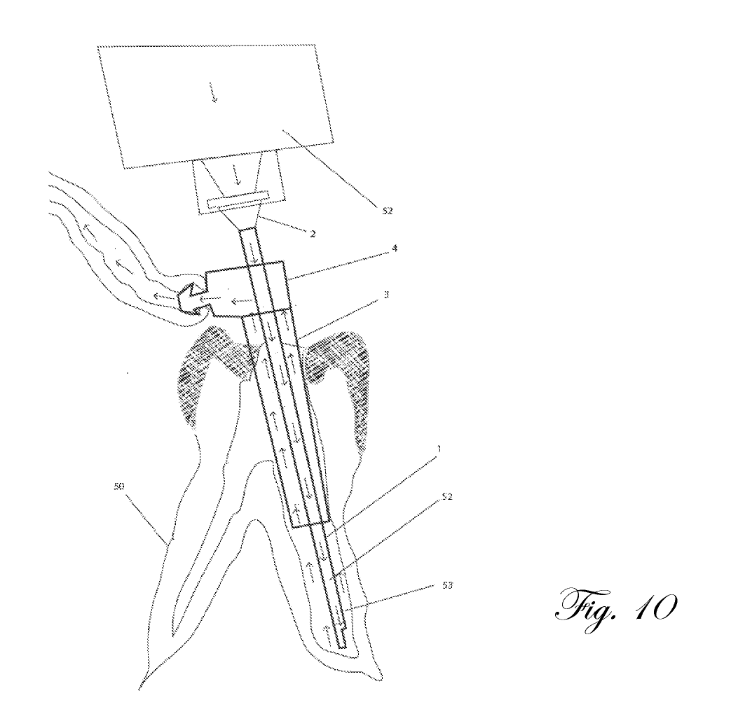

FIG. 10 is a schematic illustration of an exemplary irrigation probe and aspiration sleeve of the type shown in FIG. 1 with a syringe for use with irrigation and aspiration of caustic irrigant solution typically used in endodontic irrigation. Irrigant is expelled from the irrigation syringe or fluid source 51, the irrigant moves down the irrigation probe 1 into the root canal 52 of a tooth 50 or a wound to a skin, the irrigant circulates back up root canal or wound carrying debris 53, and the irrigant is aspirated by the aspiration sleeve 3 at the surface of the tooth or skin.

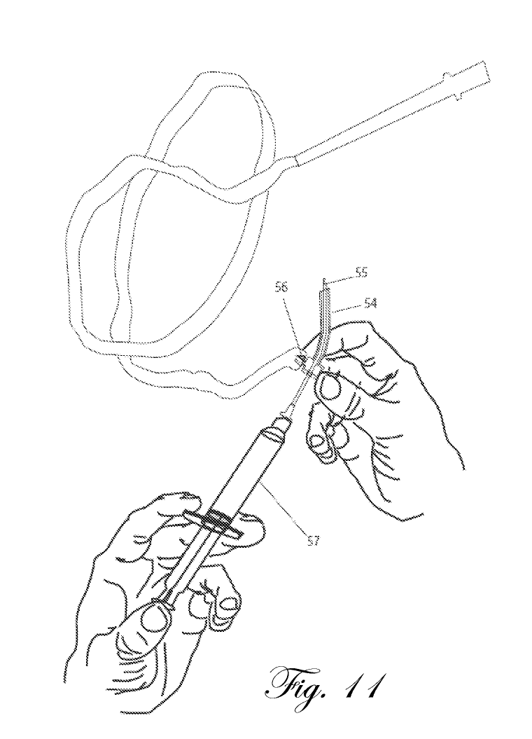

FIG. 11 is schematic illustration of an exemplary irrigation probe and aspiration sleeve of the types shown in the prior figures with a syringe 57 filled with irrigant solution held in one hand and injected, and the vacuum sheath 56 controlled with the other hand so that the relationship of the distal portion of the sheath 54 and exposed irrigant probe 55 can be controlled at the surface of the tooth as shown in FIG. 11. Thus, the operator can control the flow of irrigant fluid from the syringe 57 with one hand, and control the depth of the irrigant probe in the root canal and the position of the vacuum sheath relative to the crown of the tooth and probe with the other hand. Previous irrigant devices cannot provide this convenience and level of control.

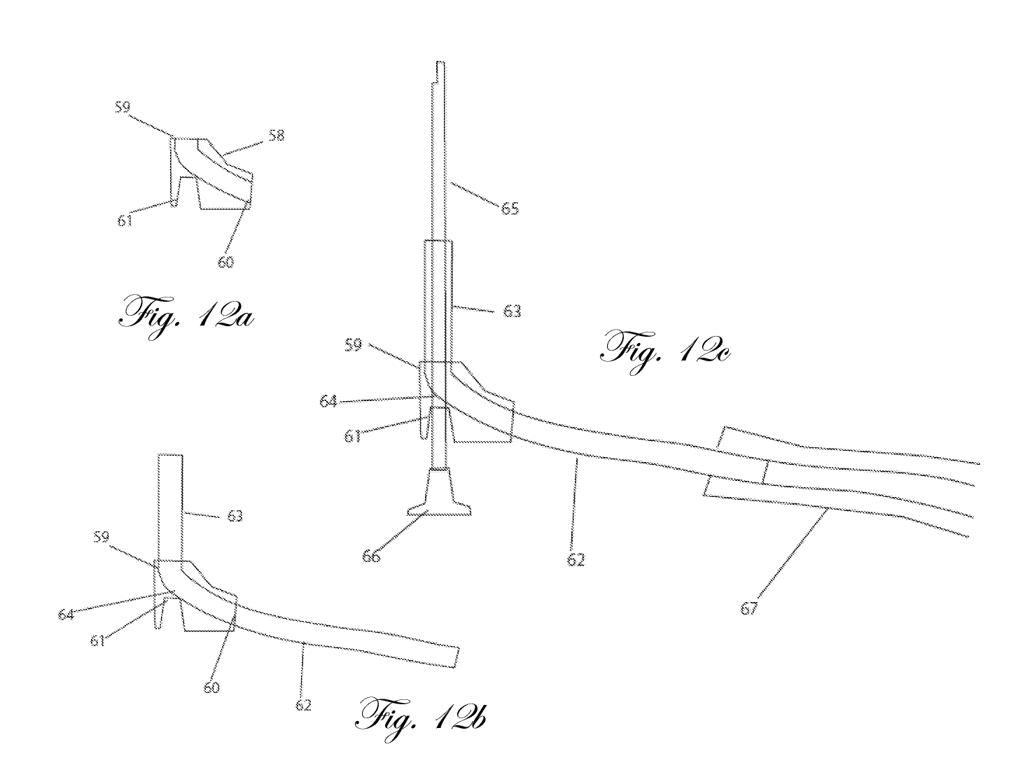

FIG. 12 is a schematic illustration of an exemplary irrigation probe and aspiration sheath of another embodiment that uses a frame to control one piece of vacuum tubing so that it can penetrated safely by the probe within minimal danger of penetration injury to the operators fingers. FIG. 12A shows the sheath frame 58 consisting of an exit port 59 that holds the tubing in a fixed or near-fixed positional distally, and an ingress port 60 that holds the tubing in a fixed or near-fixed position proximately. A depression or conical port 61 guides the needle to penetrate the tubing at a pre-determined position and markedly reduces the risk of penetrating the fingers during the process of penetrating the tubing. In FIG. 12B the tubing 62 is fitted in the frame 58 and is held in position at ingress port 60 and exit port 59. A length of tubing 63 protrudes distally from exit port 59. FIG. 12C shows that the irrigation probe 65 has been placed in the conical access port 61, penetrates the tubing at point 61, moves through exit port 59, is within the lumen of distal tubing 53, and the irrigation portion of the probe 65 protrudes out of the sheath. The proximal portion of the tubing 62 is attached to a vacuum source either directly, with a fitting, or by inserting inside a larger vacuum tubing 67. This embodiment can be used identically as shown in FIGS. 10 and 11. This device can provide similar function to that shown in U.S. Pat. No. 3,871,009 to Kahn, but with this embodiment the movement of the sheath on the probe is smoother because the needle can be bent distally rather than proximately, and the risk of accidental needlestick from the probe during assembly is markedly reduced by the funnel shaped probe port that provides controlled introduction of the needle into the tubing and the use of a frame that permits the tubing to be held firmly while the fingers are protected by the frame. Unlike U.S. Pat. No. 3,871,009 to Kahn, this device can also be mass-produced inexpensively with minimal hand assembly.

### Example Embodiments

An example irrigation and aspiration device for irrigating and aspirating a root canal of a tooth or a wound to skin according to the present invention can comprise: an irrigation probe comprising a hollow shaft having a distal end and a proximal end disposed along a substantially straight axis and having an outer perimeter, for irrigating the root canal or wound with an irrigant from a tip at the distal end of the hollow shaft; means for delivering the irrigant to the irrigation probe; a hollow aspiration sleeve that ensheathes the irrigation probe, the hollow aspiration sleeve having a distal end and a proximal end, for aspirating the root canal or wound from a suction inlet at the distal end of the hollow aspiration sleeve; and means for providing a vacuum to the hollow aspiration sleeve, wherein the outer perimeter of the proximal end of the hollow shaft of the irrigation probe makes a substantially air-tight junction with a through opening, disposed along the axis of the hollow shaft, in the vacuum providing means such that the irrigation probe is movable within the hollow aspiration sleeve; wherein the hollow shaft of the irrigation probe is collinear with the hollow aspiration sleeve along the entire length of the hollow shaft that is ensheathed by the hollow aspiration sleeve.

In an example device as in the preceding paragraph, the irrigation probe can comprise a needle, catheter, cannula, or nozzle. In an example device as in the preceding paragraph, the irrigant delivering means can comprise a fitting at the proximal end of the irrigation probe for delivery of the irrigant thereto. In an example device as in the preceding paragraph, the irrigant delivering means can comprise a handle with an on-off valve for continuous delivery of the irrigant. In an example device as in the preceding paragraph, the irrigant delivering means can comprise a syringe. In an example device as in the preceding sentence, the syringe can comprise a refilling syringe. In an example device as in the preceding sentence, the refilling syringe can comprise double one-way valves for refilling the syringe. In an example device as in the preceding paragraph, the irrigant delivering means and vacuum providing means can comprise a reciprocating syringe. In an example device as in the preceding sentence, the reciprocating syringe can comprise an accessory barrel for providing vacuum to the hollow aspiration sleeve and an injection barrel for delivery of the irrigant to the irrigation probe. In an example device as in the preceding paragraph, the vacuum providing means can comprise a vacuum housing having an air-tight bond to the proximal end of the hollow aspiration sleeve, the air-tight junction with the proximal end of the irrigation probe, and a fitting for connection of a vacuum source thereto. In an example device as in the preceding paragraph, the device can further comprise a stylet that can be inserted into and removed from the proximal end of the hollow shaft of the irrigation probe. In an example device as in the preceding paragraph, the device can further comprise a splash shield attached to the outer perimeter of the hollow aspiration sleeve to prevent splash back at an operator. In an example device as in the preceding sentence, the splash shield can be movably attached to the outer perimeter of the hollow aspiration sleeve. In an example device as in the preceding sentence, the splash shield can be transparent.

An example fitting according to the present invention for facilitating assembly of an irrigation and aspiration device, can comprise a rigid or semirigid unit having a first channel therethrough, wherein the channel enters the unit at an ingress port along a first axis and exits at an exit port along a second axis nonparallel to the first axis, wherein the first channel has an internal diameter configured to allow passage of conventional vacuum tubing, and wherein the first channel follows a path from the ingress port to the exit port that has sufficiently gentle curves to allow conventional vacuum tubing to be passed through the channel; and wherein the unit has a second channel therethrough, wherein the second channel enters and exits the unit along a third axis that is collinear with the second axis, and wherein the second channel defines an acceptance opening at the entry of the second channel into the unit, wherein the acceptance opening is shaped to facilitate insertion of an irrigation probe.

In an example fitting as in the preceding paragraph, the acceptance opening can define a funnel shape.

An example irrigation and aspiration device according to the present invention can comprise: an irrigation probe comprising a straight hollow tube having a distal end configured to output irrigant and a proximal end configured to supply irrigant to the hollow tube; a fitting comprising a rigid or semirigid member having an internal cavity and a vacuum fitting, wherein the vacuum fitting is in communication with the internal cavity and is configured to form a sealed connection to a vacuum source, and wherein the cavity defines a passageway extending along an axis, open at a first end; wherein the straight hollow tube of the irrigation probe extends through the passageway past the first end and engages the fitting in a sealed, slidable relationship along the passageway axis at a portion of the fitting opposite the first end.

The present invention has been described as an irrigation and aspiration device. It will be understood that the above description is merely illustrative of the applications of the principles of the present invention, the scope of which is to be determined by the claims viewed in light of the specification. Other variants and modifications of the invention will be apparent to those of skill in the art.

We claim:

1. An irrigation and aspiration device for irrigating and aspirating a root canal of a tooth or a wound to skin, comprising:
a. an irrigation probe comprising a hollow shaft having a distal end and a proximal end disposed along a substantially straight axis and having an outer perimeter, for irrigating the root canal or wound with an irrigant from a tip at the distal end of the hollow shaft;
b. means for delivering the irrigant to the irrigation probe;
c. a hollow aspiration sleeve that ensheathes the irrigation probe, the hollow aspiration sleeve having a distal end and a proximal end, for aspirating the root canal or wound from a suction inlet at the distal end of the hollow aspiration sleeve; and
d. means for providing a vacuum to the hollow aspiration sleeve, wherein the outer perimeter of the proximal end of the hollow shaft of the irrigation probe makes a substantially air-tight junction with a through opening, disposed along the axis of the hollow shaft, in the vacuum providing means such that the irrigation probe is movable within the hollow aspiration sleeve;
e. wherein the hollow shaft of the irrigation probe is collinear with the hollow aspiration sleeve along the entire length of the hollow shaft that is ensheathed by the hollow aspiration sleeve.

2. The irrigation and aspiration device of claim 1, wherein the irrigation probe comprises a needle, catheter, cannula, or nozzle.

3. The irrigation and aspiration device of claim 1, wherein the irrigant delivering means comprises a fitting at the proximal end of the irrigation probe for delivery of the irrigant thereto.

4. The irrigation and aspiration device of claim 1, wherein the irrigant delivering means comprises a handle with an on-off valve for continuous delivery of the irrigant.

5. The irrigation and aspiration device of claim 1, wherein the irrigant delivering means comprises a syringe.

6. The irrigation and aspiration device of claim 5, wherein the syringe comprises a refilling syringe.

7. The irrigation and aspiration device of claim 6, wherein the refilling syringe comprises double one-way valves for refilling the syringe.

8. The irrigation and aspiration device of claim 1, wherein the irrigant delivering means and vacuum providing means comprise a reciprocating syringe.

9. The irrigation and aspiration device of claim 9, wherein the reciprocating syringe comprises an accessory barrel for providing vacuum to the hollow aspiration sleeve and an injection barrel for delivery of the irrigant to the irrigation probe.

10. The irrigation and aspiration device of claim 1, wherein the vacuum providing means comprises a vacuum housing having an air-tight bond to the proximal end of the hollow aspiration sleeve, the air-tight junction with the proximal end of the irrigation probe, and a fitting for connection of a vacuum source thereto.

11. The irrigation and aspiration device of claim 1, further comprising a stylet that can be inserted into and removed from the proximal end of the hollow shaft of the irrigation probe.

12. The irrigation and aspiration device of claim 1, further comprising a splash shield attached to the outer perimeter of the hollow aspiration sleeve to prevent splash back at an operator.

13. The irrigation and aspiration device of claim 12, wherein the splash shield is movably attached to the outer perimeter of the hollow aspiration sleeve.

14. The irrigation and aspiration device of claim 12, wherein the splash shield is transparent.

15. A fitting for facilitating assembly of an irrigation and aspiration device, comprising a rigid or semirigid unit having a first channel therethrough, wherein the channel enters the unit at an ingress port along a first axis and exits at an exit port along a second axis nonparallel to the first axis, wherein the first channel has an internal diameter configured to allow passage of conventional vacuum tubing, and wherein the first channel follows a path from the ingress port to the exit port that has sufficiently gentle curves to allow conventional vacuum tubing to be passed through the channel; and wherein the unit has a second channel therethrough, wherein the second channel enters and exits the unit along a third axis that is collinear with the second axis, and wherein the second channel defines an acceptance opening at the entry of the second channel into the unit, wherein the acceptance opening is shaped to facilitate insertion of an irrigation probe.

16. A fitting as in claim 15, wherein the acceptance opening defines a funnel shape.

17. An irrigation and aspiration device comprising:
a. An irrigation probe comprising a straight hollow tube having a distal end configured to output irrigant and a proximal end configured to supply irrigant to the hollow tube;
b. A fitting comprising a rigid or semirigid member having an internal cavity and a vacuum fitting, wherein the vacuum fitting is in communication with the internal cavity and is configured to form a sealed connection to a vacuum source, and wherein the cavity defines a passageway extending along an axis, open at a first end;
c. Wherein the straight hollow tube of the irrigation probe extends through the passageway past the first end and engages the fitting in a sealed, slidable relationship along the passageway axis at a portion of the fitting opposite the first end.
